(19) United States
(12) Patent Application Publication
(10) Pub. No.: US 2010/0241231 A1
(43) Pub. Date: Sep. 23, 2010

(54) INTERVERTEBRAL FIXATION DEVICE

(76) Inventors: James F. Marino, La Jolla, CA (US); Jamil Elbana, San Diego, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/710,158

(22) Filed: Feb. 22, 2010

Related U.S. Application Data
(60) Provisional application No. 61/154,117, filed on Feb. 20, 2009.

(51) Int. Cl. A61F 2/44

(52) U.S. Cl. 623/17.15

(57) ABSTRACT

Described herein are low profile, vertebral body fixation systems that join together adjacent vertebrae to stabilize a portion of a spine and provide anterior to posterior, inferior to superior and side to side rotational control. An embodiment of the intervertebral fixation device includes an interbody element sized to be positioned between first and second adjacent vertebrae, an intravertebral body element that comprises a portion that extends into a vertebral body of the first vertebra; and a fixation element that engages the vertebral body of the first vertebra and that also engages the portion of the intravertebral body element that extends into the vertebral body of the first vertebra.

### REFERENCE TO PRIORITY DOCUMENT

This application claims the benefit of priority under 35 U.S.C. §119(e) of U.S. Provisional Application Ser. No. 61/154,117, filed Feb. 20, 2009, entitled “Intervertebral Fixation Device.” The subject matter of the above-noted application is incorporated by reference in its entirety by reference thereto.

### BACKGROUND

An intervertebral disc may be subject to degeneration caused by trauma, disease, and/or aging. Problems created by disc degeneration can be treated by partial or full discectomy with or without spinal fusion. For example, anterior cervical discectomy with fusion (ACDF) is a common procedure for treating cervical degenerative disc disease. Partial or full removal of the degenerated disc can destabilize the spinal column and reduce a natural spacing between adjacent vertebrae. Reduced spacing between adjacent vertebrae may increase pressure on nerves that pass between vertebral bodies causing pain and/or nerve damage.

Given these post-operative problems, implants, cages or other prostheses can be inserted into the intervertebral disc space of the neighboring vertebral bodies after removal of a damaged disc or pulposus of a disc. Insertion of such devices can restore the original height of the disc and the original distance between the two neighboring vertebral bodies. Further, natural movements of the neighboring vertebral bodies relative to each other can be maintained including the freedom of movement in flexion and extension of the vertebral bodies.

In the example of ACDF, the goal of the spinal fusion is to successfully “grow” two or more vertebrae together either by bone or other material placed between the vertebrae in the disc space and/or over posterior elements of the vertebrae. The majority of ACDFs fuse, but there are still some that go on to non-union. Post-operative site motion following fusion procedures can result in non-unions. Motion can be avoided using spinal instrumentation, including cages, placed onto or into the spine to immobilize the vertebrae that are going to be fused. These devices provide the best possible environment for successful arthrodesis and recovery.

Ideally, interbody implants should restore disk height, place the annular fibers in a “normal” tension, create lordosis through the joined vertebral bodies, obtain sagittal balance through the segment, reduce subluxed facet joints, enlarge the neuroforaminal space, and restore to normal the proportion of weight bearing through the anterior spinal column.

Interbody implants known in the art are generally stabilized only by an interference fit. Such implants rely on precise sizing and ligament tension for stability. Plates can be used with such interbody implants to improve stability and further limit motion between the upper and lower vertebra. Plate fixation, however, provides only a tension band effect in one direction. For example, an anterior plate limits extension whereas flexion, in contrast, is poorly restrained due to the relatively thin and pliable anterior plate. Further, anterior plate fixation devices known in the art use screws that purchase only the anterior cortex and/or the relatively weak cancellous bone of the adjacent vertebral body. This results in the screws being vulnerable to loosening and backing out.

### SUMMARY

There is a growing need for improved methods and devices for linking tissue structures in an animal body. In particular, there is a need for a low profile, vertebral body fixation system that joins together adjacent vertebrae to stabilize a portion of a spine and provide anterior to posterior, inferior to superior and side to side rotational control.

In an embodiment, disclosed is an intervertebral fixation device having an interbody element sized to be positioned between first and second adjacent vertebrae, an intravertebral body element that comprises a portion that extends into a vertebral body of one of the adjacent vertebrae and a fixation element that engages the vertebral body of the vertebra and also a portion of the intravertebral body element that extends into the vertebral body of the vertebra.

The device can further comprise a second intravertebral body element having a second portion that extends into the second vertebral body of the second vertebra. The first vertebra can be a superior vertebra and the second vertebra can be an inferior vertebra. The interbody element can contact an endplate of at least one of the first and second adjacent vertebrae. The fixation element can purchase a cortical margin of at least one of the first and second vertebrae. The portion of the intravertebral body element that extends into a vertebral body of the first vertebra can include at least one fenestration. The fixation element can engage the portion of the intravertebral body element through the fenestration. The intravertebral body element can include a second portion available to engage the interbody element from within the disc space. The second portion can include a rail and the interbody element can include a track that is linearly translatable along the rail. Linear translation of the interbody element along the rail can approximate the first and second adjacent vertebrae. The interbody element can include a bore that extends generally posterio-anteriorly through the interbody element. At least a portion of the bore can intersect with at least a portion of the track. The device can further include a locking element inserted through the bore that engages the rail linearly translated in the track. The interbody element can be an interbody cage. The device can further include an anterior plate having at least one bore, the bore having a screw head recess. The interbody element can include superior and inferior surfaces that are generally non-parallel. The interbody element can include one or more cavities that extend from superior to inferior vertebral endplates. The one or more cavities in the interbody element can include bone graft or osteoconductive material.

Also disclosed is a method of treating a spinal disorder with an intervertebral fixation device described herein. The method includes cutting a first and second trough in a first vertebral endplate of a first vertebra adjacent an intervertebral disc space to be treated, wherein the first and second troughs are on opposing sides of a spinal midline; advancing an intravertebral body element into the intervertebral disc space to be treated; inserting a first portion of the intravertebral body element into the first trough; and translating an interbody element into the intervertebral space such that it mates with the first vertebral endplate of the first adjacent vertebra and with a second vertebral endplate of a second adjacent vertebra.

The method can also include transfixing with a first fixation element the first portion of the intravertebral body element inserted through the first trough, the fixation element also engaging a cortex of the first adjacent vertebra. The method can also include transfixing with a second fixation element a first portion of a second intravertebral body element inserted through the second trough, the fixation element also engaging a cortex of the second adjacent vertebra. The interbody element can include generally non-parallel superior and inferior surfaces. Translating the interbody element into the intervertebral disc space can include restoring lordotic angulation. The spinal midline can include a plane that bisects the first vertebral endplate along an axis of insertion.

Other features and advantages will be apparent from the following description of various embodiments, which illustrate, by way of example, the principles of the disclosed devices and methods.

### BRIEF DESCRIPTION OF THE DRAWINGS

FIG. 1A is a simplified sagittal view of a vertebrae pair;

FIG. 1B is a simplified, sectional axial view of a vertebra;

FIG. 2A illustrates a perspective view of an embodiment of an intervertebral fixation device inserted between a vertebrae pair;

FIGS. 2B-2E illustrate perspective view of the intervertebral fixation device of FIG. 2A step-wise inserted between a vertebrae pair;

FIG. 3A illustrates a perspective view of an embodiment of intravertebral body projecting elements;

FIGS. 3B-3E illustrate various views of an embodiment of an interbody element;

FIGS. 4A-4D illustrate the insertion of an interbody element into an embodiment of an intravertebral body projecting element;

FIGS. 5A-5C illustrate an embodiment of a threaded locking element of the device;

FIG. 6 illustrates another embodiment of an intervertebral fixation device;

FIGS. 7A-7F illustrate another embodiment of an intervertebral fixation device;

FIGS. 8A-8J illustrate the implantation and assembly of an embodiment of an intervertebral fixation device.

### DETAILED DESCRIPTION

Throughout this description, embodiments and variations are described for the purpose of illustrating uses and implementations of the devices and systems. The illustrative description should be understood as presenting examples, rather than as limiting the scope of the subject matter.

The intervertebral fixation devices described herein secure or transfix an intervertebral device or element by connecting a relatively rigid member engaged into at least one cortex of an adjacent vertebra to another element mechanically associated with the intervertebral device, such that the engagement or linkage occurs within the volume defined by the vertebral body and/or intervertebral space. In an embodiment, the rigid member can be a screw that is fixed to the cortex of a vertebra and screwed into the interbody device within the intervertebral space or a mechanically linked element projecting from the intervertebral body into the substance of the vertebra. The intervertebral fixation devices described herein provide stability in both flexion and extension as well as in lateral bending and between inferior and superior vertebrae.

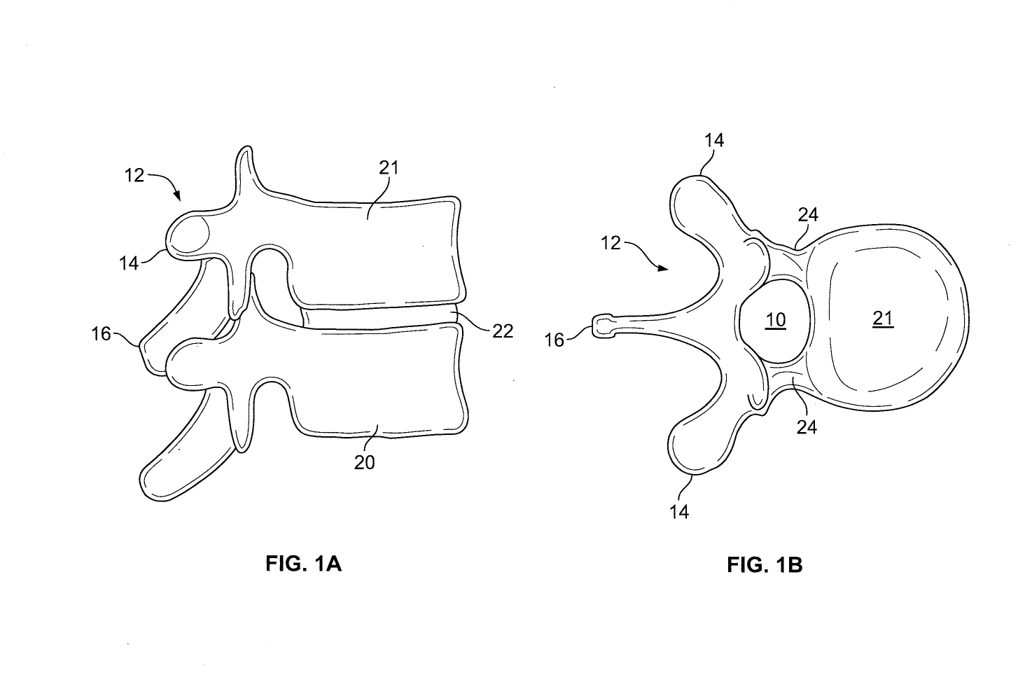

FIG. 1A is a simplified sagittal view of a vertebrae pair 20, 21. FIG. 1B is a simplified, sectional axial view of the vertebrae 21 of the vertebrae pair shown in FIG. 1A. Each vertebra 20, 21 includes lamina 12, transverse processes 14, a spinous process 16, central canal 10, and pedicles 24. A disc 22 comprised of an annulus and disc nucleus (not shown) is located between the vertebrae pair 20, 21 where the vertebrae pair 20, 21 and disc 22 form a coupled articulated jointed bony interface.

An intervertebral implant, such as the intervertebral fixation devices described herein, may be used to restore and stabilize a portion of a spine following partial or full removal of a disc. Intervertebral implants can restore a normal separation distance between vertebrae adjacent to the degenerated disc and can restore increased cross-sectional area and volume to the central canal, lateral recess, and neuroforamina.

Intervertebral implants can be used in combination with one or more plates to provide further support and stability, for example an anterior plate fixation system. Anteroposterior movement in a human spine may include flexion and extension. Anterior plates known in the art provide only a tension band effect and are therefore of limited effect in resisting flexion moments. Flexion moments are poorly restrained due to a relatively thin and pliable plate located anterior to the center of rotation. In addition, resection of the posterior longitudinal ligament, often performed during disc decompression, further contributes to construct instability. The intervertebral fixation devices described herein provide stabilizing support in flexion, extension, as well as in lateral bending.

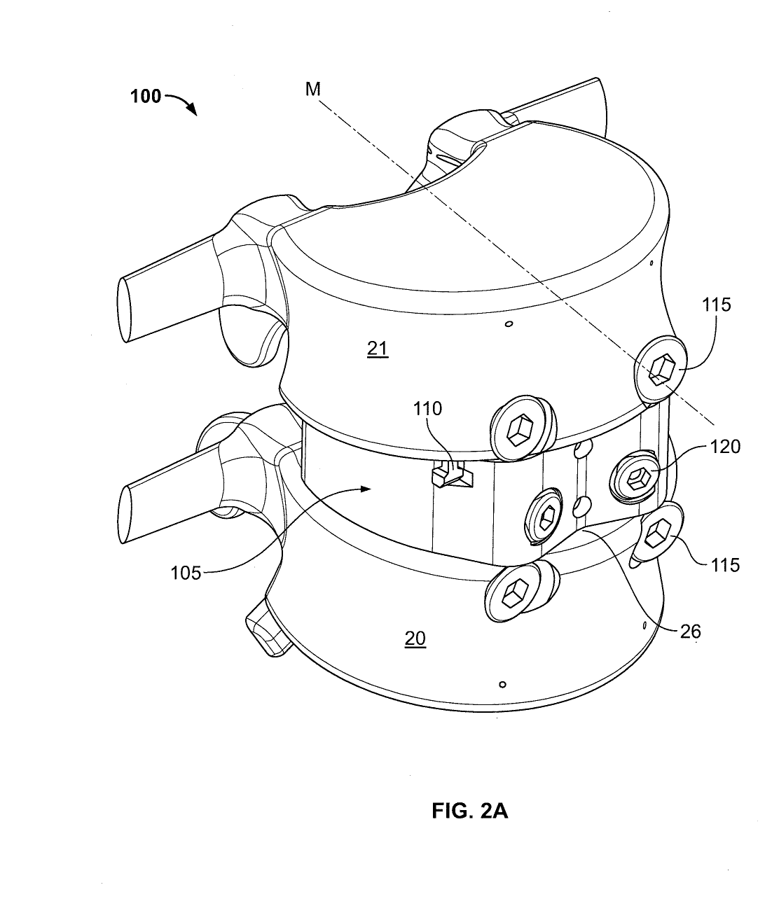

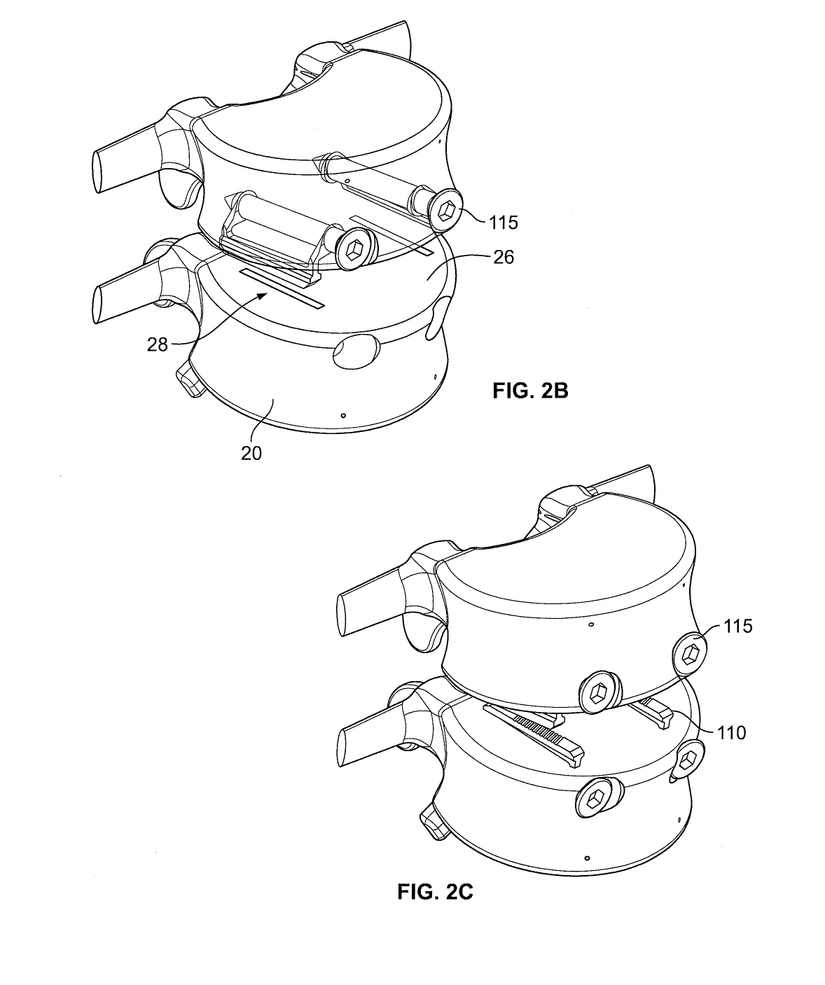

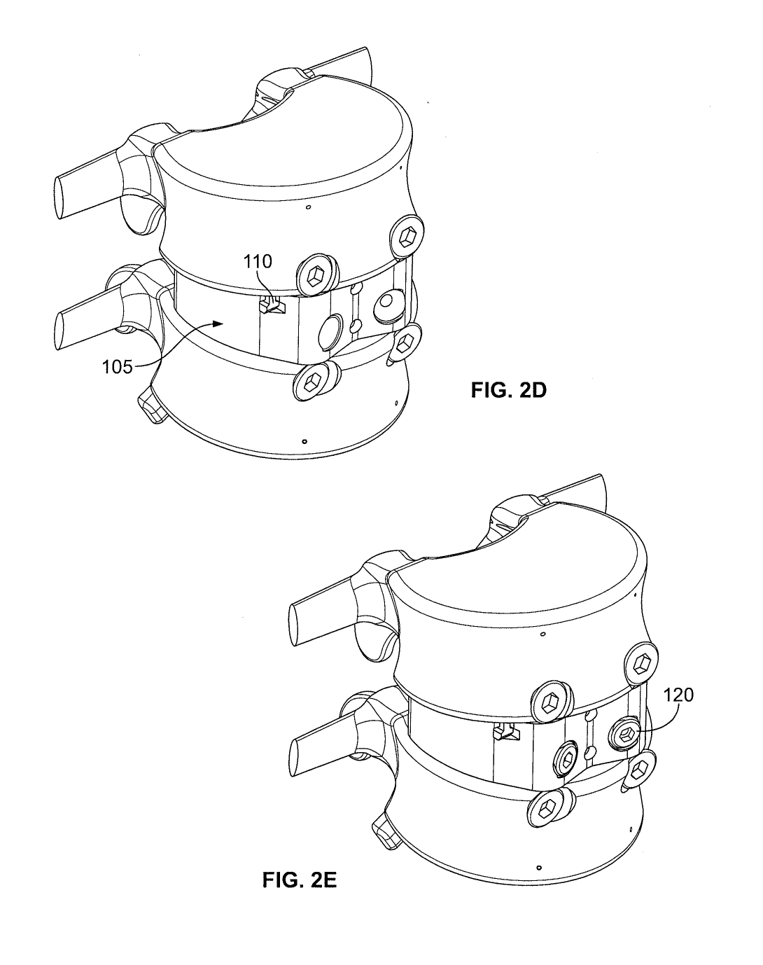

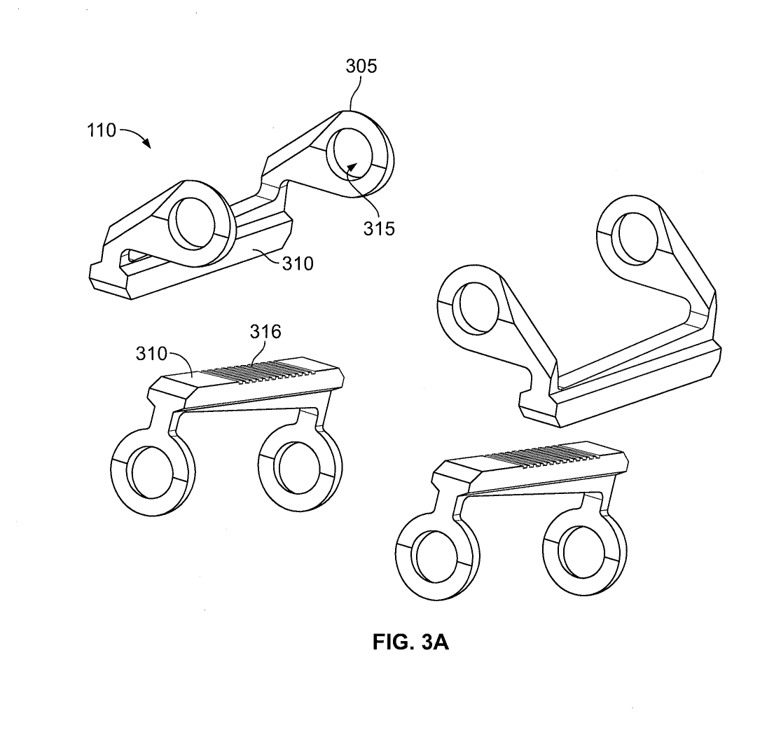

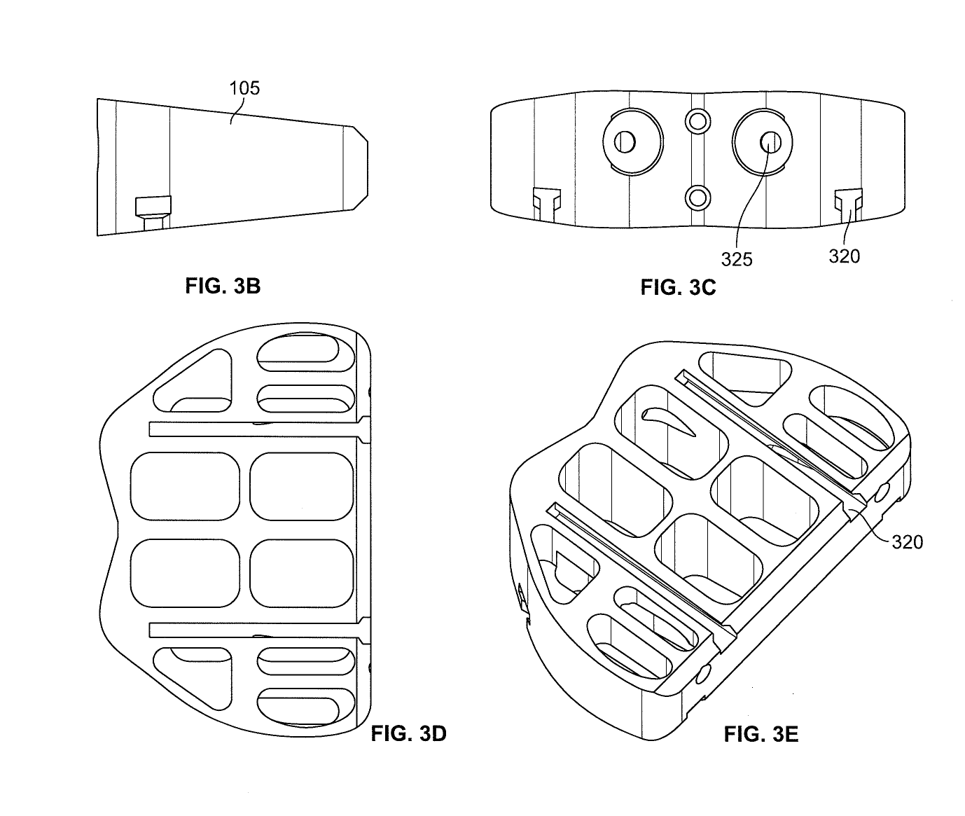

FIGS. 2A-2E show perspective views of an embodiment of an intervertebral fixation device 100. The device 100 generally includes at least one interbody element 105, at least one intravertebral body projecting element 110 (see FIG. 3A), at least one cortex fixation element 115 and at least one assembly locking element 120. The interbody element 105 can be positioned between an inferior and superior vertebra 20, 21 within the intervertebral disc space, for example, vacated after discectomy. The interbody element 105 can be an intervertebral cage or spacer. The intravertebral body projecting elements 110 engage the interbody element 105 as well as the vertebrae. The cortex fixation elements 115 engage the intravertebral body projecting elements 110 as well as the cortex of the vertebrae. In turn, the intravertebral body projecting element 110 in combination with the cortex fixation elements 115 transfix the interbody element 105 to the inferior and superior vertebrae 20, 21.

In the embodiment shown in FIGS. 2A-2E the interbody element 105 contacts the endplates 26 of the inferior and superior vertebrae 20, 21. In an embodiment, the endplates 26 can be prepared prior to implantation of the device, such as for example by cutting one or more narrow troughs 28 into the vertebral body (see FIG. 2B). In an embodiment, the trough 28 is formed generally along the posterior aspect of the inferior and/or superior vertebral bodies 20, 21. In another embodiment, the trough 28 is cut through the endplate 26 on one or both sides of the spinal midline M shown in the figures as a plane that bisects the associated vertebra along an axis of implant insertion. The trough 28 can be approximately (5 mm to 10 mm) millimeters deep and (3 mm to 10 mm) millimeters wide such that it can receive a portion of the intravertebral body projecting element 110 as described in more detail below.

As shown in FIG. 3A, portion 305 of the intravertebral body projecting elements of 110 are inserted into prepared troughs in the vertebral body. In an embodiment, the portion 305 of the intravertebral body projecting element 110 that extends into the vertebral body can include a fenestration 315.

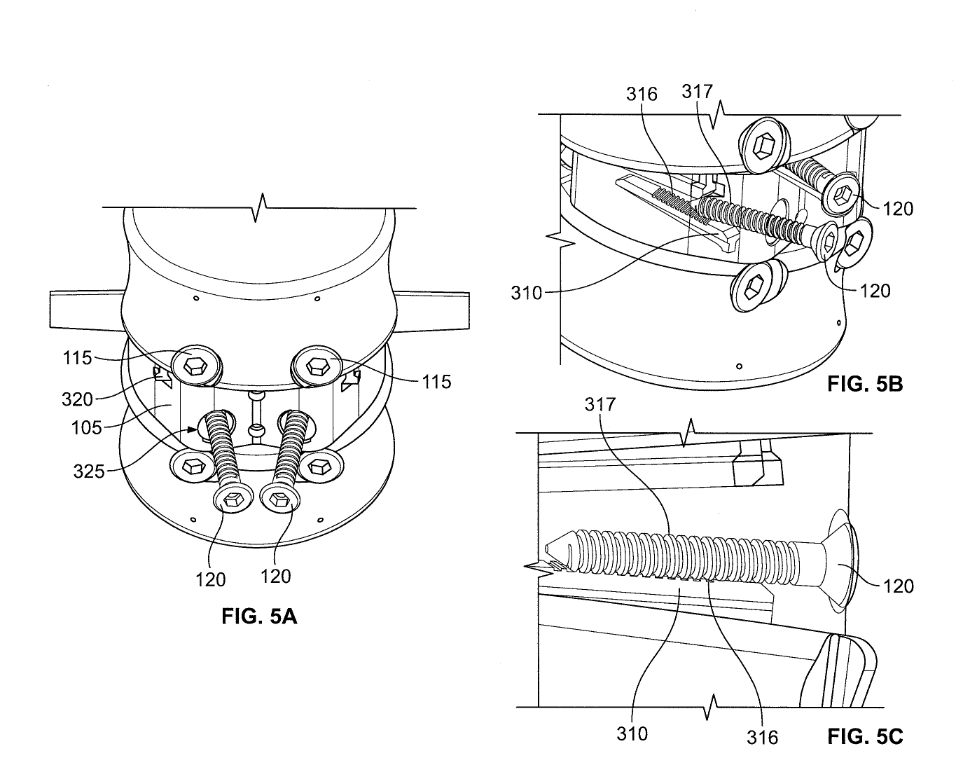

Still with respect to FIG. 3A, the intravertebral body projecting elements of 110 can also include a portion 310 that remains accessible from the vacated intervertebral space. In one embodiment, this portion 310 of the intravertebral body projecting element 110 can function as a rail or track system that engages one or more cavities or tracks 320 present in the interbody element 105 (See FIG. 3B-3E). In another embodiment, portion 310 can have a threaded surface 316 that engages the threads 317 of the one or more assembly locking elements 120 as described in more detail below (see FIG. 5B).

As best shown in FIG. 3B, the interbody element 105 has generally non-parallel superior and inferior surfaces. These surfaces mate with the adjacent prepared vertebral endplates. The shape of the interbody element 105 can vary. In an embodiment, the interbody element 105 can be wedge-shaped. The result can be a restoration or maintenance of lordotic angulation. The interbody element 105 can also have cavities extending between its inferior and superior surfaces providing a “swiss cheese” type appearance (see FIG. 3E). This can create abundant relative volume, for example for bone graft material, while providing a large surface area for prepared vertebral endplates to contact and provide for osteoconductive healing. The interbody element 105 as well as any of the components described herein can be composed of various materials, including radiolucent plastics or polymers such as PEEK, and/or relatively inert implantable materials such as titanium and titanium alloys.

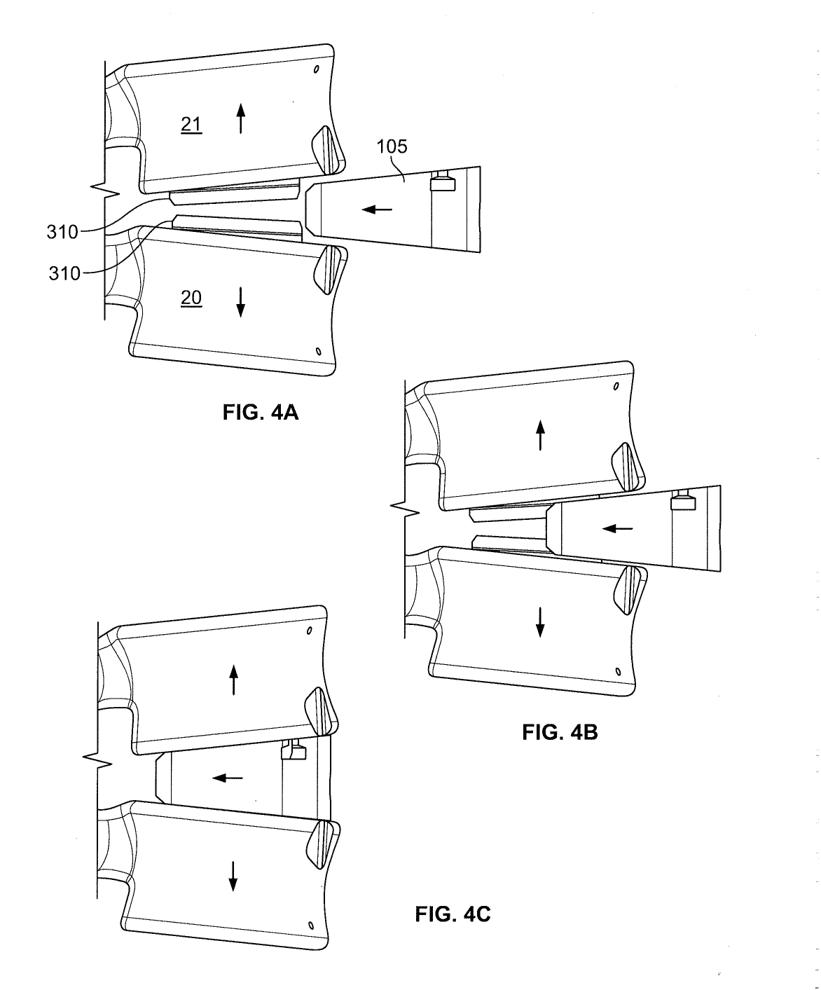

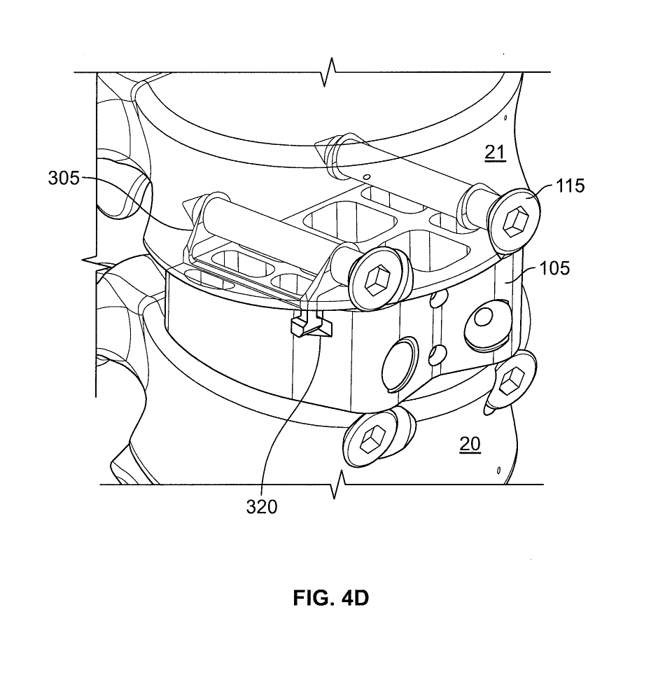

Still with respect to the embodiment shown in FIGS. 3B-3E, the interbody element 105 can include one or more tracks 320 on either or both of its inferior or superior surfaces. In an embodiment, the interbody element 105 has at least one track 320 on its superior surface and at least one track 320 on its inferior surface. In an embodiment, the interbody element 105 has two tracks 320 each on opposite sides of the spinal midline M. The track(s) 320 of the interbody element 105 can allow for translation along the rail portion 310 of the intravertebral body projecting element 110 (see FIGS. 4A-4C). The interbody element 105 can be translated along the rail portion 310 from an insertion to a resting position. The linear translation movement of the interbody element 105 posteriorly results in an approximation of the adjacent vertebral bodies 20, 21. In an embodiment, the interbody element 105 has at least one track 320 on its superior surface and at least one track 320 on its inferior surface that engage with their respective superior and inferior rails 310 of intravertebral body projecting elements 110 affixed to the vertebrae pair.

Still with respect to FIGS. 3B-3E, the interbody element 105 also can include one or more bores 325 that are accessible from its anterior surface and configured to receive a locking element 120 through an anterior opening (see also FIG. 5A). At least a portion of the bore 325 intersects with at least a portion of the track 320 such that threading locking element 120 through the bore 325 locks the intervertebral fixation device 100, as discussed in more detail below.

As mentioned above, the intervertebral fixation device 100 includes at least one cortex fixation element 115. Plate fixation devices known in the art employ screws that purchase the anterior cortex only and the relatively weak cancellous bone of the adjacent vertebral body. This makes them vulnerable to loosening and backing out (therefore requiring mechanisms to prevent screw retreat). The one or more cortex fixation elements 115 of the intervertebral fixation device 100 described herein engage and transfix the intravertebral body projecting element 110 (and thereby the interbody element 105) as well as at least one cortex of an adjacent vertebra. The one or more cortex fixation elements 115 can engage the intravertebral body projecting element 110 through the fenestration 315 of portion 305 (see FIG. 4D). In an embodiment, the one or more cortex fixation elements 115 can engage the anterior cortex of the vertebrae. In another embodiment, the one or more cortex fixation elements 115 can engage the posterior vertebral cortex. In another embodiment, the one or more cortex fixation elements 115 can engage the lateral cortex or lateral cortexes of a vertebral body. In a further embodiment, the one or more cortex fixation elements 115 extend through the endplate of the adjacent vertebrae. In an embodiment, the cortex fixation element 115 is a screw, such as a pedicle screw.

Now with respect to the embodiment shown in FIGS. 5A-5C, the device 100 can include one or more threaded locking elements 120. Locking element 120 can be inserted through bore 325 in interbody element 105. The bore 325 has an opening at the anterior surface of the interbody element 105. At least a portion of bore 325 intersects at least a portion of an adjacent track 320 such that as locking element 120 is inserted through bore 325 and the threads 317 of the locking element 120 engage the threaded surface 316 of rails 310 of intravertebral body projecting elements 110 affixed to the vertebra. In an embodiment the device 100 includes an interbody element 105 having two tracks 320 each on opposite sides of the spinal midline M and two bores 325 each on opposite sides of the spinal midline M such that at least a portion of each bore 325 intersects at least a portion of its respective track 320.

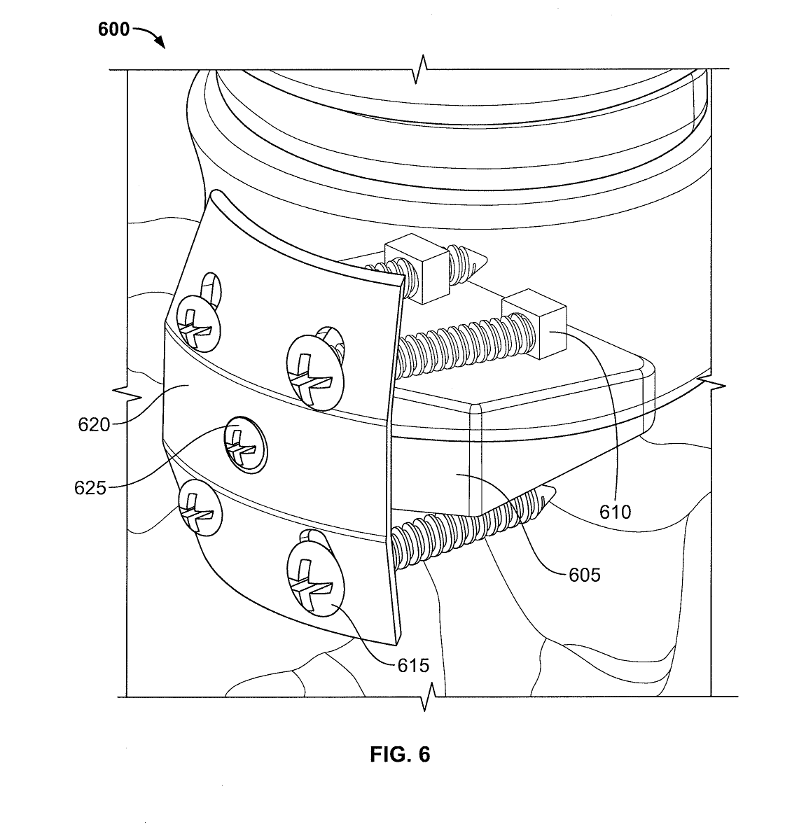

It should be appreciated that the interbody element 105 and the intravertebral body projecting elements 110 do not necessarily connect using a rail and track engagement. FIG. 6 shows another embodiment of an intervertebral fixation device 600. In this embodiment, the fixation device 600 includes at least one interbody element 605, at least one intravertebral body projecting element 610, at least one fixation element 615 and, alternatively, an anterior plate 620. As in the previous embodiment, the interbody element 605 can be positioned between an inferior and superior vertebra 20, 21 within the vacated intervertebral disc space, for example, after discectomy. The intravertebral body projecting elements 610 engage the interbody element 605 as well as the vertebrae. The fixation elements 615 engage the intravertebral body projecting elements 610 as well as the cortex of the vertebrae. In turn, the intravertebral body projecting element 610 in combination with the fixation elements 615 transfix the interbody element 605 between the inferior and superior vertebrae 20, 21.

The interbody element 605 contacts the endplates of the inferior and superior vertebrae 20, 21. The endplates 26 can be previously prepared, such as for example by cutting one or more narrow troughs into the vertebral body. The intravertebral body projecting element 610 can project posteriorly into the trough cut into a vertebral body and vacated disc space. The one or more fixation elements 615 insert through the anterior plate 620 (if present), through the vertebral cortex and engage the intravertebral body projecting element 610 of the interbody element 605.

The plate 620 can provide enhanced stability and still maintain a low anterior profile. The plate 620 can have screw head recess 625 that provides anterior dynamic compression with final screw advancement. In an embodiment, the plate 620 marries an anterior surface feature (not shown) on the interbody element 605.

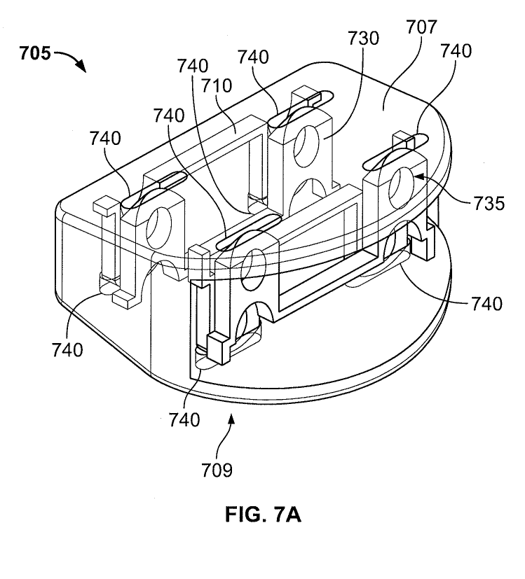

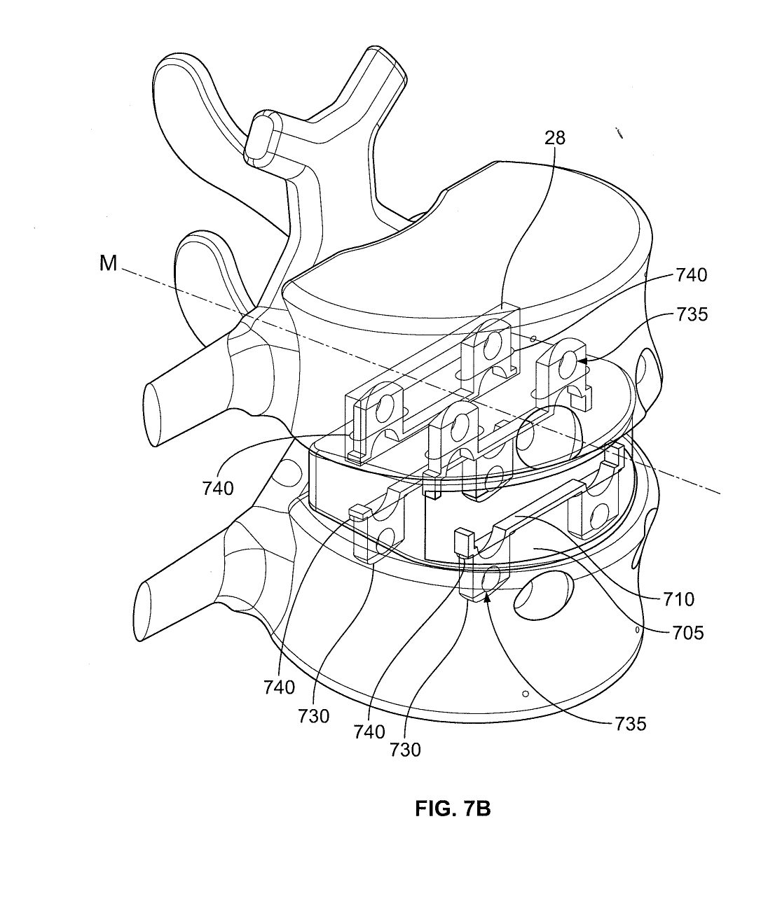

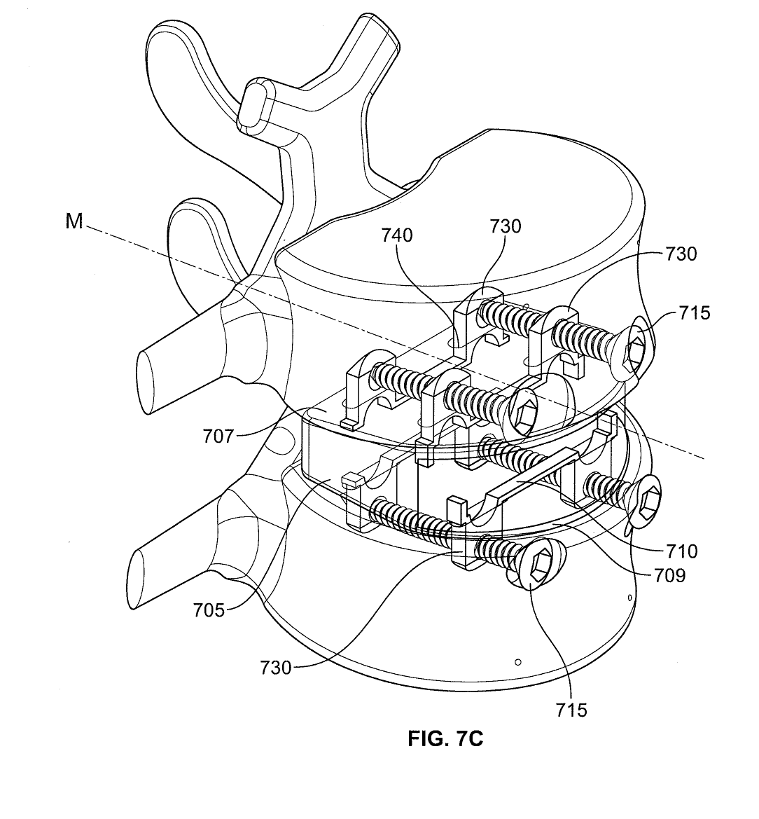

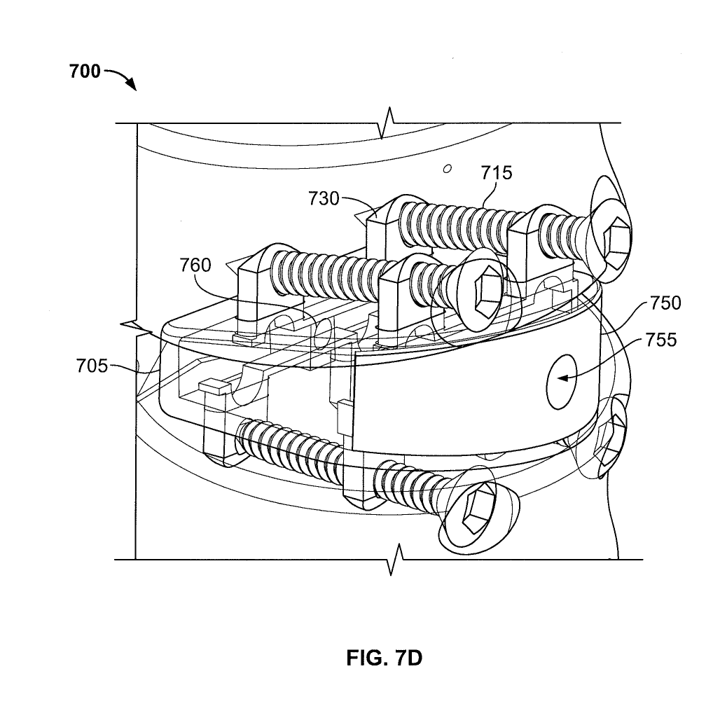

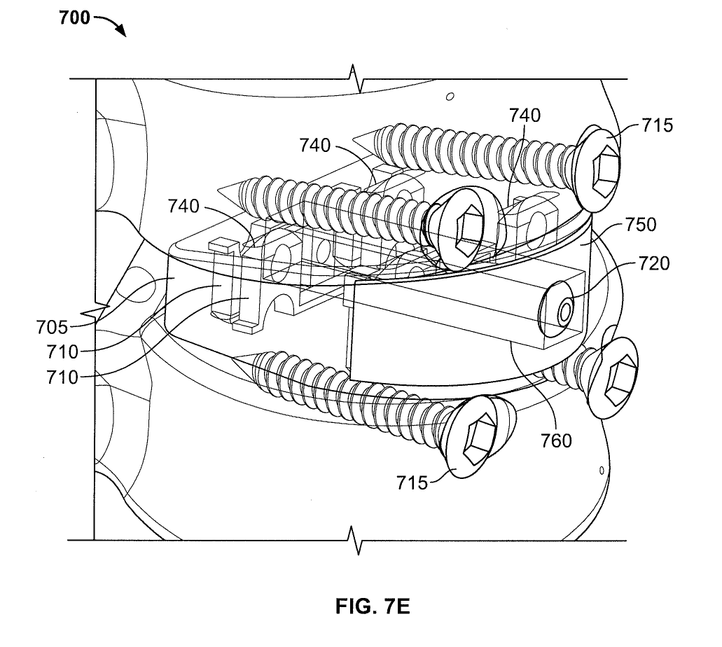

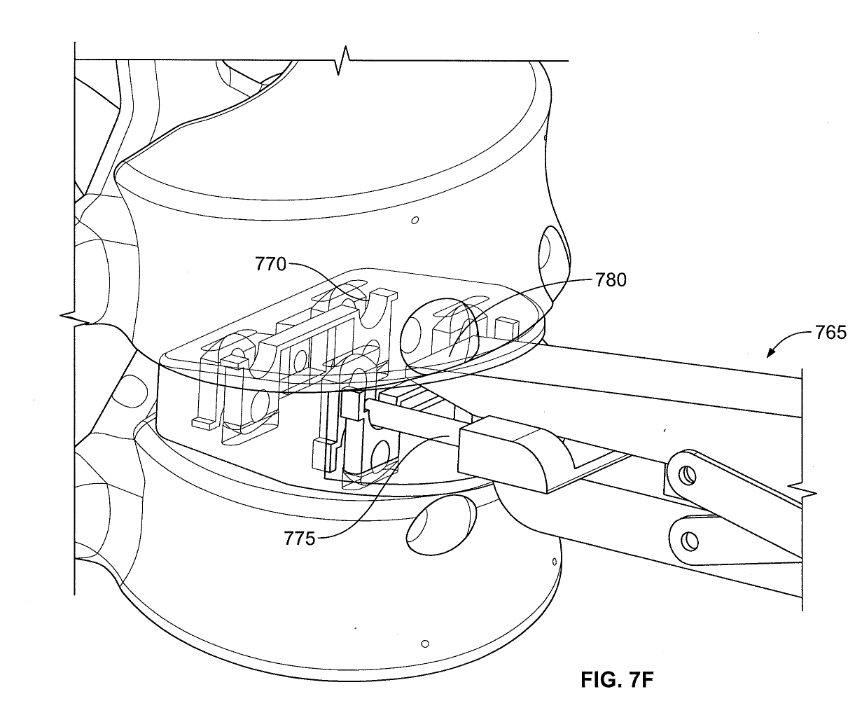

FIGS. 7A-7F illustrate another embodiment of the intervertebral fixation device 700. The intervertebral fixation device 700 can have intravertebral body projecting elements 710 contained within an interbody element 705 prior to insertion between the vertebral pair. This embodiment also does not rely on a rail and track engagement mechanism between the interbody element 705 and the projecting elements 710.

The device 700 generally includes an interbody element 705, intravertebral body projecting elements 710, cortex fixation elements 715 and an assembly locking element 720. As mentioned, the intravertebral body projecting elements 710 are wholly contained within the interbody element 705 prior to insertion between the vertebral pair (see FIG. 7A). Once the interbody element 705 is positioned between the vertebrae within the intervertebral disc space, at least a portion of the intravertebral body projecting elements 710 can be delivered through one or more slots 740 in the interbody element 705 such that the portion inserts through the prepared endplates of the vertebral pair. The intravertebral body projecting elements 710 engage the interbody element 705 as well as the vertebral bodies. The interbody element 705 is essentially hollow and includes abundant relative volume for bone graft material or other osteoconductive healing material to be inserted therein while still providing a large surface area for prepared vertebral endplates to contact and for healing to occur.

FIG. 7F illustrates an embodiment of an insertion device 765 for deploying the intravertebral body projecting elements 710 through the slots 740 in the interbody element 705. The insertion device 765 has deployment members 775, 780 that can be inserted anteriorly through the interbody element 705 and engage the intravertebral body projecting element 710 to press the intravertebral body projecting element 710 through the slots 740 in the interbody element 705 thereby deploying them within the trough of the endplate of neighboring vertebrae. In an embodiment, the insertion device 765 can be actuated such that deployment member 775 engages a feature 770 of the intravertebral body projecting element 710. Feature 770 can have a surface geometry that is complementary to the surface geometry of the deployment member 775. The deployment member 780 presses against the superior portion of the interbody element 710 as deployment member 775 pushes downward on feature 770 thereby forcing intravertebral body projecting element 710 through the slot 740 and into the trough 28 of the endplate.

The slots 740 can be located on either or both of the superior 707 and inferior 709 surfaces of interbody element 705. The slots 740 can be sized and shaped for at least a portion of an intravertebral body projecting element 710 to extend therethrough. The slots 740 can be located on either side of the vertebral midline M. In an embodiment, a slot 740 is located generally along the posterior aspect of the superior 707 and inferior 709 surfaces of the interbody element 705 and on either side of the vertebral midline M. A slot 740 can also be located generally along the anterior aspect of the superior 707 and inferior 709 surface of the interbody element 705 on either side of the vertebral midline M.

The endplates of the vertebral pair can be prepared as described in other embodiments such that troughs 28 are formed. The troughs can generally align with the slots 740 in the interbody element 705 (see for example FIG. 7B). One or more troughs can be formed in the vertebral endplates that are shaped and sized to receive at least a portion of an intravertebral body projecting element 710 projecting through the one or more slots 740 in the interbody element 705. The trough can be formed generally through the endplates of each of the vertebral pair such that each receives at least a portion of an intravertebral body projecting element 710 on either side of the vertebral midline M. In an embodiment, a trough is formed generally along the posterior aspect of the superior and inferior vertebral bodies on either side of the vertebral midline M and another trough is formed along the anterior aspect of the superior and inferior vertebral bodies on either side of the vertebral midline M such that each trough can receive at least a portion of an intravertebral body projecting element 710 in each of the vertebral bodies.

Portion 730 of the intravertebral body projecting elements 710 projects through the slots 740 in the interbody element 705 and inserts into the prepared troughs in the vertebral bodies. In an embodiment, the portion 730 includes a fenestration 735. A cortex fixation element 715 can engage the fenestration 735 of the intravertebral body projecting elements 710 and the cortex of the vertebrae. The intravertebral body projecting element 710 in combination with the cortex fixation element 715 transfixes the interbody element 705 between the vertebral pair.

As shown in FIGS. 7D-7E, the device 700 can also include a plate 750 fixed near the anterior region of the interbody element 705. The anterior plate 750 is fixed by an assembly locking element 720 that inserts through an aperture 755 in the plate 750 and engages bore 760 running generally between the anterior and posterior regions of the interbody element 705.

The configuration of the devices 100, 600, 700 described herein provides an “I-beam” effect. The devices 100, 600, 700 provide tension to the projecting elements 110, 610, 710 that are engaged by the fixation elements 115, 615, 715 advanced through the interbody elements 105, 605, 705 (and a plate 620, 720, if present). The result is a relatively rigid, locked “box” linkage for anterior to posterior, superior to inferior, and side to side rotational control. The devices 100, 600, 700 also can provide compaction of any bone graft material or osteoconductive material used.

It should be appreciated that the intervertebral fixation devices 100, 600, 700 can include more than one of each of the interbody elements 105, 605, 705 intravertebral body projecting elements 110, 610, 710, fixation elements 115, 615, 715 or locking elements 120, 620, 720. The intervertebral fixation devices described herein 100, 600, 700 can each also include a plate. In an embodiment, the fixation devices 100, 600, 700 include one, two, three, four or more intervertebral body projecting elements 110, 610, 710 that extend into the vertebral bodies of one or both of the superior and inferior vertebras adjacent to an intervertebral disc space. In another embodiment, the fixation devices 100, 600, 700 includes one, two, three, four or more fixation elements 115, 615, 715 that traverse at the vertebral cortex. In an embodiment more than one vertebral cortex is transfixed by each fixation device 100, 600, 700.

### Methods of Use

It should also be appreciated that each component of the fixation devices 100, 600, 700 described herein can be inserted anteriorly or laterally. In preferred embodiments, all the components of the fixation devices 100, 600, 700 can each be inserted using an anterior approach. An anterior approach may result in less muscle and tissue damage than many lateral posterior stabilization approaches. Further, employing a single direction approach prevents the need for a patient to be flipped during the operation thereby reducing the risk of surgical complications.

It should also be appreciated that embodiments of the fixation devices 100, 600, 700 described herein can be used for cervical, thoracic as well as lumbar applications. In some embodiments, an intervertebral fixation device can be used to replace a disc in the lumbar region of a spine. In some embodiments, an intervertebral fixation device can be used in the cervical or thoracic regions of a spine. Embodiments of the fixation devices 100, 600, 700 described herein can be used independently or in conjunction with other systems or devices to provide stability to the spine. Implantation of embodiments of the fixation devices 100, 600, 700 described herein can be implanted using minimally invasive techniques, with only minimal intrusion to adjacent tissue and muscle.

### Implantation and Assembly of Intravertebral Fixation Devices

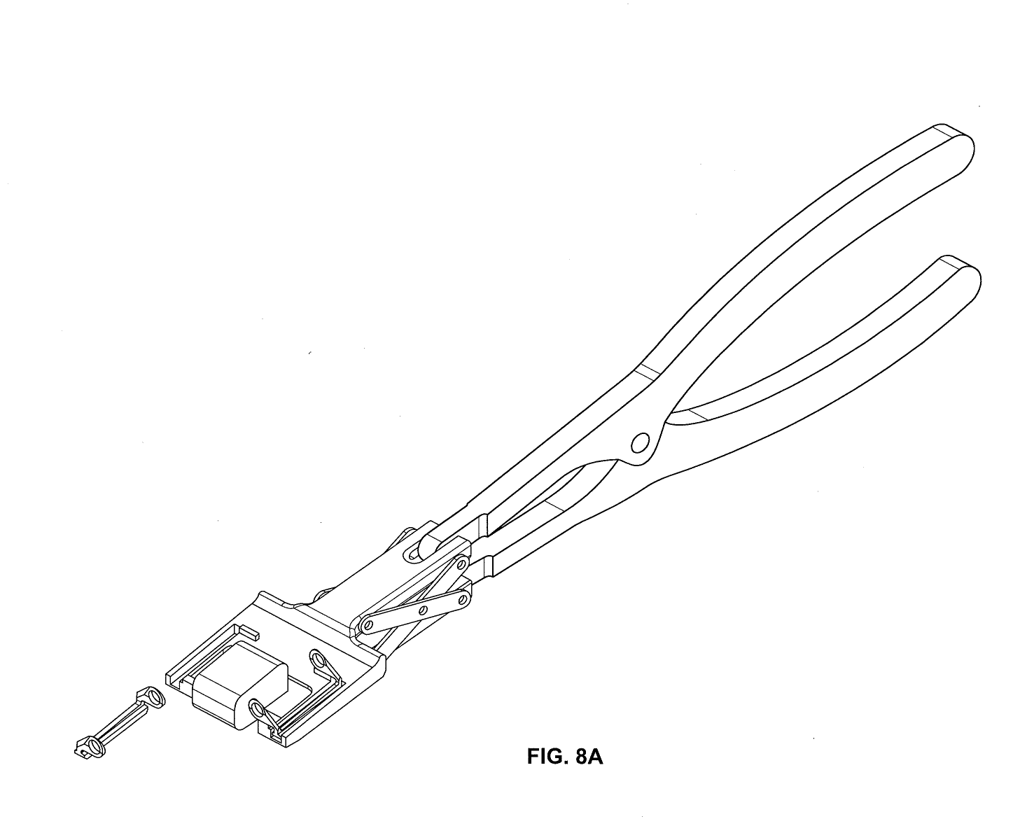

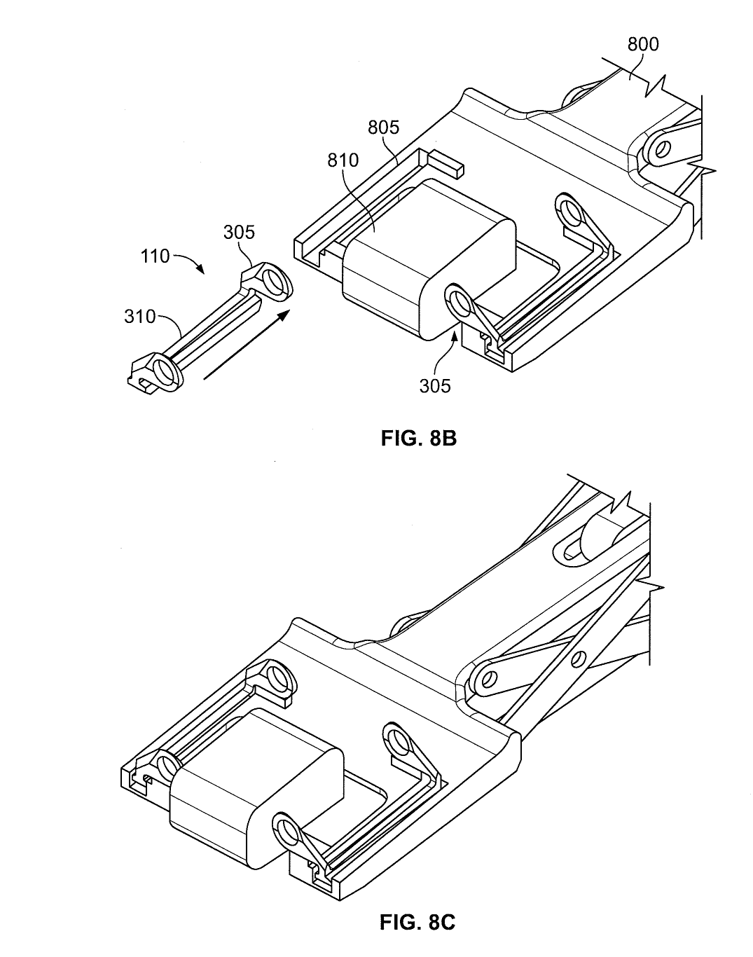

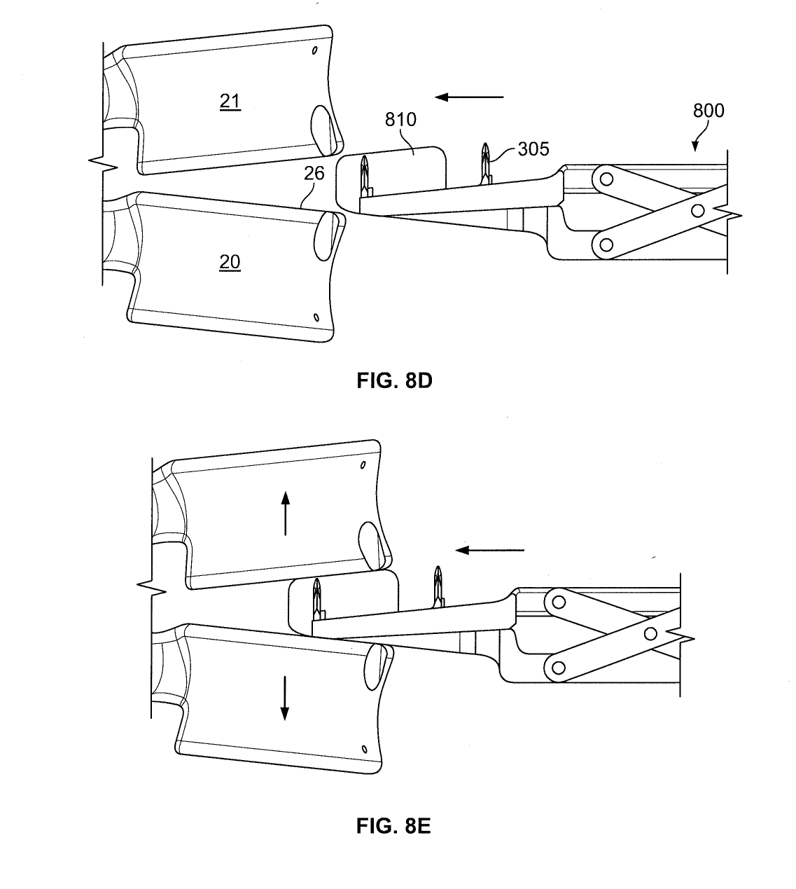

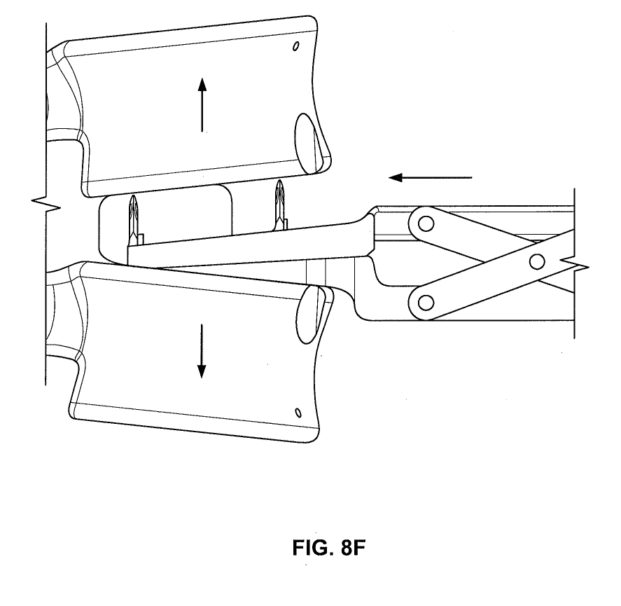

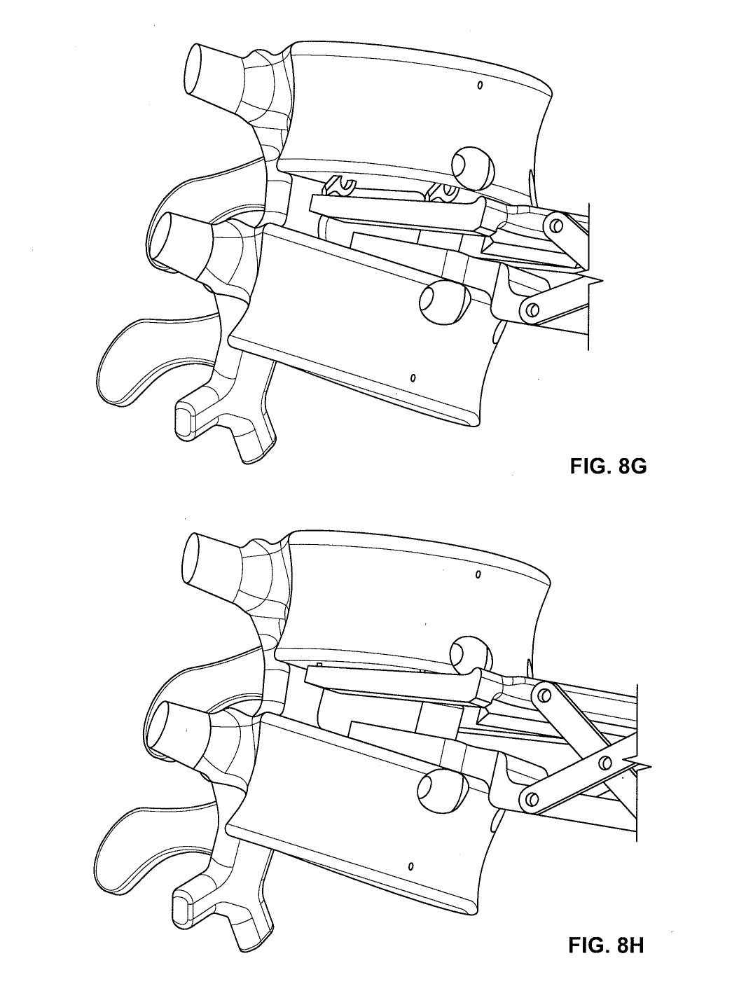

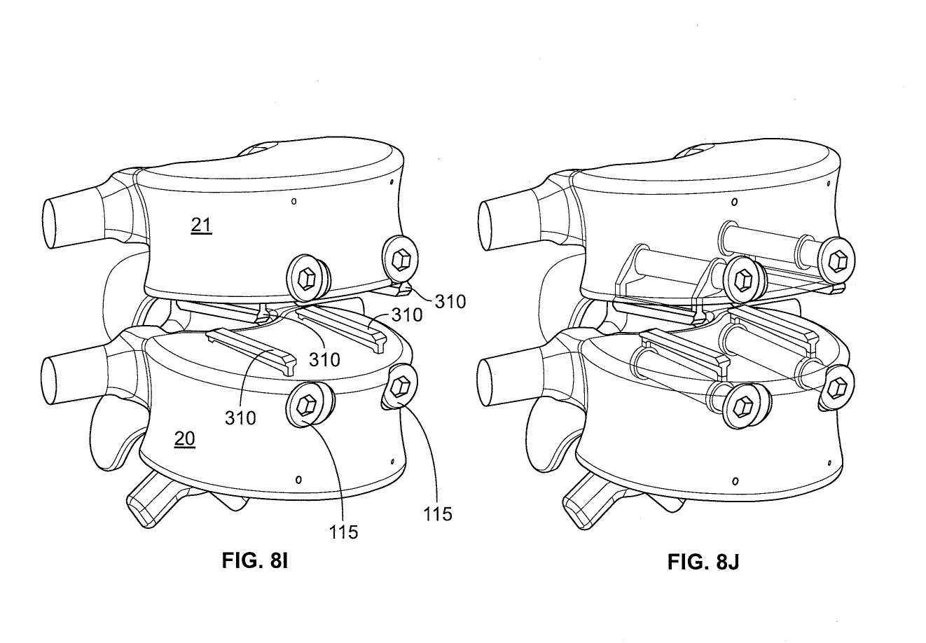

FIGS. 8A-8J illustrate the implantation and assembly of an embodiment of an intervertebral fixation device. Following preparation of the vertebral endplate 26 as known in the art, the intravertebral body projecting elements 110 can be inserted into the troughs 28. As shown in FIGS. 8A-8C, each of the projecting elements 110 are loaded onto an inserter 800 such as by sliding the rail portion 310 into corresponding tracks 805 in the inserter 800 leaving the fenestrated portion 305 available for insertion into the trough 28 of the prepared vertebral endplate 26.

The inserter 800 loaded with the intravertebral body projecting elements 110 can be wedged between the vertebrae within the prepared disc space as shown in FIGS. 8D-8F. A wedge feature 810 is located on the leading edge of the inserter 800 to separate the inferior and superior vertebrae from one another and provide space for insertion of the intravertebral body projecting elements 110. FIGS. 8G-8H illustrate the insertion of fenestrated portion 305 of the intravertebral body projecting element 110 into the trough 28 formed in the superior vertebral endplate. FIG. 8I-8J show the superior and inferior vertebral endplates each with an inserted fenestrated portion 305 of the intravertebral body projecting element 110 into the trough 28. The rail portions 310 of the intravertebral body projecting elements 110 are accessible from the intervertebral disc space on either side of the midline of the superior and inferior vertebrae. Also shown are the cortex fixation elements 115 inserted through both the vertebral cortex and the fenestration portion 305 of the intravertebral body projecting element 110.

Looking back at FIGS. 4A-4D, an interbody element 105 having superior tracks 320 on each side of the spinal midline and inferior tracks 320 on each side of the spinal midline is translated along the rail system 310 of the intravertebral body projecting elements 110 affixed to the vertebrae pair. The linear translation movement of the interbody element 105 in a posterior direction results in an approximation of the adjacent vertebral bodies 20, 21.

Looking again at FIGS. 5A-5C, the interbody element 105 is locked into place upon insertion of threaded locking elements 120 through bore 325 such that the threads 317 engage with the threaded surface 316 of the rail 310 system 310 affixed to the vertebrae pair.

It should be appreciated that the devices herein do not necessarily connect using a rail and track engagement. For example, the embodiment of FIGS. 7A-7F would differ somewhat in the method of implantation and assembly as described above.

While this specification contains many specifics, these should not be construed as limitations on the scope of any embodiment that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Only a few examples and implementations are disclosed. Variations, modifications and enhancements to the described examples and implementations and other implementations may be made based on what is disclosed.

What is claimed is:

1. An intervertebral fixation device, comprising:
an interbody element sized to be positioned between first and second adjacent vertebrae;
an intravertebral body element that comprises a portion that extends into a vertebral body of the first vertebra; and
a fixation element that engages the vertebral body of the first vertebra and that also engages the portion of the intravertebral body element that extends into the vertebral body of the first vertebra.

2. The device of claim 1, further comprising a second intravertebral body element comprising a second portion that extends into the second vertebral body of the second vertebra.

3. The device of claim 2, wherein the first vertebra comprises a superior vertebra and the second vertebra comprises an inferior vertebra.

4. The device of claim 1, wherein the interbody element contacts an endplate of at least one of the first and second adjacent vertebrae.

5. The device of claim 1, where the fixation element purchases a cortical margin of at least one of the first and second vertebrae.

6. The device of claim 1, wherein the portion of the intravertebral body element that extends into a vertebral body of the first vertebra comprises at least one fenestration.

7. The device of claim 6, wherein the fixation element engages the portion of the intravertebral body element through the fenestration.

8. The device of claim 1, wherein the intravertebral body element comprises a second portion available to engage the interbody element from within the disc space.

9. The device of claim 8, wherein the second portion comprises a rail and wherein the interbody element comprises a track that is linearly translatable along the rail.

10. The device of claim 9, wherein linear translation of the interbody element along the rail approximates the first and second adjacent vertebrae.

11. The device of claim 10, wherein the interbody element comprises a bore that extends generally posterio-anteriorly through the interbody element.

12. The device of claim 11, wherein at least a portion of the bore intersects with at least a portion of the track.

13. The device of claim 12, further comprising a locking element inserted through the bore that engages the rail linearly translated in the track.

14. The device of claim 13, wherein the interbody element comprises an interbody cage.

15. The device of claim 1, further comprising an anterior plate, wherein the plate comprises at least one bore, the bore having a screw head recess.

16. The device of claim 1, wherein the interbody element comprises superior and inferior surfaces and wherein the superior and inferior surfaces are generally non-parallel.

17. The device of claim 1, wherein the interbody element comprises one or more cavities that extend from superior to inferior vertebral endplates.

18. The device of claim 17, wherein the one or more cavities in the interbody element include bone graft or osteoconductive material.

19. A method of treating a spinal disorder in a patient with an intervertebral fixation device, comprising:
cutting a first and second trough in a first vertebral endplate of a first vertebra adjacent an intervertebral disc space to be treated, wherein the first and second troughs are on opposing sides of a spinal midline;
advancing an intravertebral body element into the intervertebral disc space to be treated;
inserting a first portion of the intravertebral body element into the first trough; and
translating an interbody element into the intervertebral space such that it mates with the first vertebral endplate of the first adjacent vertebra and with a second vertebral endplate of a second adjacent vertebra.

20. The method of claim 19, further comprising transfixing with a first fixation element the first portion of the intravertebral body element inserted through the first trough, the fixation element also engaging a cortex of the first adjacent vertebra.

21. The method of claim 20, further comprising transfixing with a second fixation element a first portion of a second intravertebral body element inserted through the second trough, the fixation element also engaging a cortex of the second adjacent vertebra.

22. The method of claim 19, wherein the interbody element comprises generally non-parallel superior and inferior surfaces.

23. The method of claim 19, wherein translating the interbody element into the intervertebral disc space comprises restoring lordotic angulation.

24. The method of claim 19, wherein the spinal midline comprises a plane that bisects the first vertebral endplate along an axis of insertion.
